(19) United States
(12) Patent Application Publication
(10) Pub. No.: US 2009/0263324 A1
(43) Pub. Date: Oct. 22, 2009

(54) Animal Model With Induced Arrhythmia

(76) Inventors: Atsushi Sugiyama, Yamanashi (JP); Akira Takahara, Chiba (JP); Yoshioki Satoh, Yamanashi (JP)

Correspondence Address: WENDEROTH, LIND & PONACK, L.L.P., 1030 15th Street, N.W.,, Suite 400 East, Washington, DC, 20005-1503, US

(21) Appl. No.: 11/885,675

(22) PCT Filed: Oct. 27, 2006
(86) PCT No.: PCT/JP2006/322044

§ 371 Date: Sep. 25, 2007

(30) Foreign Application Priority Data
Oct. 28, 2005 (JP) 2005-315434

(51) Int. Cl. A61B 10/00; A01K 67/027

(52) U.S. Cl. 424/9.2; 800/9

(57) ABSTRACT

Provided are a model animal physiologically similar to humans, enabling a highly reproducible evaluation of the onset of drug-induced long QT syndrome, a method of generating the same, and an evaluating method using the same. A proarrhythmia model animal of a monkey, prepared by ablating the atrioventricular node; the foregoing monkey is preferably a cynomolgus monkey. A method of generating a proarrhythmia model animal, comprising a step for inserting an electrode catheter to the heart of a monkey, and ablating atrioventricular node with the catheter, and a method of evaluating the QT interval prolongation by a drug, comprising using the foregoing model animal.

### TECHNICAL FIELD

The present invention relates to an arrhythmia model animal having a mechanism of onset similar to that of humans. Specifically, the present invention relates to a model animal that enables an evaluation of the QT interval prolongation by a drug, a method of preparing the same, a method of evaluation using the same and the like.

### BACKGROUND ART

It has been reported that drugs, other than antiarrhythmic drugs, in actual use in clinical settings sometimes prolong electrocardiogram QT interval and induce fatal ventricular arrhythmia called Torsades de pointes (TdP). Sudden death of a person who has been in ordinary social life represents a major damage not only to his or her family, but also to society and economy.

It had been difficult to predict the onset of drug-induced long QT syndrome, which occurs only in particular patients, from the results of conventional nonclinical studies using normal animals. As a result, drugs possessing proarrhythmic action had been prescribed for susceptible patients in clinical settings, resulting in the above-described worst case of arrhythmic death occurring frequently all over the world. To avoid such cardiac events due to onset of drug-induced long QT syndrome, the ICH (The International Conference on Harmonization of Technical Requirements for Registration of Pharmaceuticals for Human Use) signed S7B (Guideline for Nonclinical Evaluation of Potential Possibility of Pharmaceuticals for Human Use for Ventricular Repolarization Delay (QT Interval Prolongation)) and E14 (Guideline for Clinical Evaluation of Potential Possibility of Non-antiarrhythmic Drugs for QT/QTc Interval Prolongation and Arrhythmogenic Action) as Step 4 (ICH Harmonized Tripartite Guideline Final Agreements) in May 2005, and definitely described the roles of nonclinical studies. For example, it was newly prescribed that conduct of an S7B study must be considered before the test drug is administered to humans for the first time, that if the test drug is strongly positive in hERG (human ether-a-go-go related gene) and in vivo studies in S7B, though it is negative in Thorough QT/QTc (ThQT) study, the mechanism must be explained, that ThQT study can be reduced on the basis of the results of S7B study and early clinical study, and the like. Based on these facts, the notation that nonclinical study data and Phase I data per a strict protocol can substitute for ThQT study is emerging in Japan. From now on, it is anticipated that through the drug development processes, from nonclinical studies to clinical studies, integrated risk assessment will be required. To cope with this situation, understanding of the features of individual models used in nonclinical studies and accurate interpretation of the study results obtained would be a premise.

Currently, some model animals are known as heart disease models. An example atrial fibrillation model is the aconitine model (Moe et al., Am. Heart. J. 58: 59-70 (1959)), in which atrial fibrillation of topical origin is induced by topically administering aconitine to the atrial appendage, but has no direct relevance to paroxysmal atrial fibrillation in clinical settings. The aseptic pericarditis model (Page et al., J. Am. Coll Cardiol. 8: 872-879 (1986)) is a model in which induction of atrial arrhythmia is facilitated by aseptically spreading talc powder over the atrial muscle surface to cause pericarditis; Kumagai et al. demonstrated that atrial fibrillation was induced in this model. This model is used to explore the mechanism of onset of atrial fibrillation.

Japanese Patent Kokai Publication No. 2002-291373 discloses a method of generating a heart failure model animal, comprising simultaneously starting coronary arterial stenosis and stenosis of arteries other than the coronary artery and the abdominal aorta in an animal such as a dog or a rat. On the other hand, Japanese Patent Kohyo Publication No. 2002-543812 discloses a method of generating a model animal, comprising inducing ventricular arrhythmia that can cause sudden cardiac death by making an atrioventricular block and myocardial infarction in the heart of a dog. Also, the present inventors established a method of evaluation enabling prediction of the onset of drug-induced secondary long QT syndrome by using in combination two experimental models, i.e., a halothane-anesthetized dog and a chronic atrioventricular block dog (Atsushi Sugiyama, Folia Pharmacol. Jpn. 121, 393-400 (2003)). However, these model animals were highly likely to die if arrhythmia or heart failure developed.

### DISCLOSURE OF THE INVENTION

It is an object of the present invention to provide a model animal physiologically similar to humans, enabling a highly reproducible evaluation of the onset of drug-induced long QT syndrome, a method of generating the same, and a method of evaluation using the same.

The present inventors, in view of the above-described problems, diligently investigated with the aim of establishing a model using the monkey, whose heart is morphologically similar to that of humans, and which is pharmacokinetically most closely related to humans, succeeded in generating an arrhythmia model enabling an evaluation of drug-induced long QT syndrome, and developed the present invention. Accordingly, the invention of this application provides:

[1] A proarrhythmia model animal of a monkey, which is generated by ablating the atrioventricular node.
[2] The model animal of [1] above, wherein the atrioventricular node is blocked.
[3] The model animal of [1] above, wherein the ablation is conducted by electrical stimulation from the tip of a catheter.
[4] The model animal of any one term of [1] to [3] above, which is an acute phase model less than 1 month after ablation.
[5] The model animal of any one term of [1] to [3] above, which is a chronic phase model 1 month or more after ablation.
[6] The model animal of [5] above, which is a chronic heart failure model.
[7] The model animal of [6] above, wherein the concentration of atrial natriuretic peptide or cerebral natriuretic peptide in the blood is elevated compared to a normal monkey.
[8] The model animal of any one term of [1] to [5] above, which is a model of sympathetic hypertonia.
[9] The model animal of [8] above, wherein the concentration of noradrenaline in the blood is elevated compared to a normal monkey.
[10] The model animal of any one term of [1] to [9] above, wherein the monkey is a cynomolgus monkey.
[11] A method of generating a proarrhythmia model animal, comprising a step for inserting an electrode catheter to the heart of a monkey, and ablating the atrioventricular node with the catheter.
[12] The generating method of [11] above, wherein the size of the catheter is 5 to 6 French.
[13] The generating method of [11] or [12] above, wherein the monkey is a cynomolgus monkey.
[14] A method of evaluating the QT interval prolongation by a drug, comprising using the model animal of any one term of [1] to [10] above.
[15] A method of evaluating the QT interval prolongation by a drug, comprising:
a step for administering the drug to the model animal of any one term of [1] to [10] above,
a step for measuring the QT interval or QTc interval in the recipient animal, and comparing the same with the QT interval or QTc interval in the same animal before administration, and
a step for evaluating the potential possibility of the QT interval or QTc interval prolongation by the drug on the basis of the results obtained in the comparison step.
[16] A screening method for a candidate substance possessing antiarrhythmic action, comprising using the model animal of any one term of [1] to [10] above.
[17] A screening method for a candidate substance that ameliorates chronic heart failure, comprising using the model animal of [6] or [7] above.
[18] A screening method for a candidate substance that ameliorates sympathetic hypertonia, comprising using the model animal of [8] or [9] above.
[19] A proarrhythmia model animal of a monkey, wherein the is monkey possesses an atrioventricular block, and the concentration of atrial natriuretic peptide or cerebral natriuretic peptide in the blood is elevated compared to a normal monkey.
[20] The model animal of [19] above, wherein the concentration of atrial natriuretic peptide or cerebral natriuretic peptide in the blood is elevated about 2 to 50 times compared to a normal monkey.
[21] The model animal of [19] or [20] above, wherein the concentration of noradrenaline in the blood is elevated compared to a normal monkey.
[22] The model animal of [21] above, wherein the concentration of noradrenaline in the blood is elevated about 1.5 to 5 times compared to a normal monkey.
[23] The model animal of [21] or [22] above, which is a model of sympathetic hypertonia.
[24] The model animal of [19] above, which is a model concurrently suffering cardiac hypertrophy and cardiac dilation that accompany volume overload.
[25] The model animal of any one term of [19] to [24] above, wherein the monkey is a cynomolgus monkey.

### BRIEF DESCRIPTION OF THE DRAWINGS

FIG. 1 shows example electrocardiograms observed during the generation of the proarrhythmia model of the present invention. FIG. 1A shows a body surface electrocardiogram (ECG, upper panel) and intracardiac electrocardiogram recorded from an electrode at the tip of an ablation catheter (His, lower panel) before conduct of atrioventricular node ablation. FIG. 1B shows body surface electrocardiograms before and after conduct of atrioventricular node ablation.

FIG. 2 shows the results of an examination of the shape of the heart during the generation of the proarrhythmia model of the present invention. FIG. 2A shows chest radiographs before and after conduct of atrioventricular node ablation. FIG. 2B is a graph showing changes in cardiothoracic ratio (CTR). A comparison between normal monkeys (Normal) and chronic atrioventricular block monkeys (CAVB).

FIG. 3 is a graphic representation summarizing neurohumoral changes in the proarrhythmia model of the present invention. Comparisons between normal monkeys (Normal) and chronic atrioventricular block monkeys (CAVB). In the figure, * mark indicates a statistically significant difference (P<0.05), and ** mark indicates a statistically significant difference (P<0.01).

FIG. 4 shows the results of electrophysiological evaluations of the proarrhythmia model of the present invention in the acute phase and the chronic phase. FIG. 4A shows typical examples of body surface electrocardiogram (ECG) and monophasic action potential (MAP) in the acute phase and the chronic phase just after conduct of atrioventricular node ablation. FIG. 4B is a graphic representation summarizing monophasic action potential duration (MAP90), effective refractory period (ERP) and action potential terminal period (TRP) for each pacing cycle length in the acute phase and the chronic phase.

FIG. 5 shows the results of electrocardiography with administration of dl-sotalol to the proarrhythmia model of the present invention. FIG. 5A shows a typical example of electrocardiogram changes due to dl-sotalol. FIG. 5B shows a summary of the action of sotalol on electrocardiogram QTc.

FIG. 6 shows arrhythmia (Torsades de pointes (TdP)) that developed with administration of dl-sotalol to the proarrhythmia model of the present invention. FIG. 6A is a Holter electrocardiogram for the onset of arrhythmia with dl-sotalol administration. FIG. 6B is a magnified view of the portion where arrhythmia was recorded. FIG. 6C shows the number of animals having arrhythmia observed after administration of each dose.

### BEST MODE FOR EMBODYING THE INVENTION

The present invention provides a proarrhythmia model animal of a monkey. The model animal of the present invention is obtained by ablating the atrioventricular node. Also, the model animal of the present invention may be a monkey spontaneously suffering an atrioventricular block. Whether or not the monkey showing an atrioventricular block can be used as the model animal of the present invention can be determined on the basis of the electrophysiological characteristics and the concentrations of ANP, BNP and noradrenaline in the blood described below.

In the present invention, a monkey is not subject to limitation, as long as it is a monkey that can be utilized as a laboratory animal; a cynomolgus monkey, a rhesus monkey, a green monkey, a squirrel monkey, a marmoset, a tamarin and the like can be mentioned, but from the viewpoint of possible reduction in the amount of evaluation subject drug used, a cynomolgus monkey, which has a small body, is preferable.

In the present invention, proarrhythmia refers to polymorphic ventricular tachycardia that occurs when a drug known to induce arrhythmia in humans is administered at a dose about 1 to 10 times the maximum clinical daily dose; it ceases spontaneously in some cases, but in other cases it can progress to ventricular fibrillation, and eventually even to death, without spontaneous ceasing.

In the present invention, ablation refers to making electrical stimulation from the tip of a catheter, specifically to applying a high-frequency electric current from the tip of an electrode catheter to electrically cauterize the tissue in contact with the tip. The tissue to be targeted is the atrioventricular node. Because the excitation of the sinoatrial node is prevented from being transmitted to the ventricle by thus destroying the atrioventricular node to make a complete atrioventricular block, the right ventricle and the left ventricle subsequently fulfill blood pumping function by the rhythms of the His bundle and Purkinje's fiber. Hence, the pump function for pumping the blood from the heart decreases and the heart rate decreases remarkably, so that the heart is overloaded and, as a result, the entire heart hypertrophies.

The model animal of the present invention can also be a model concurrently suffering cardiac hypertrophy and cardiac dilation that accompany volume overload.

The model animal of the present invention is roughly divided into the acute phase model less than 1 month after ablation, and the chronic phase model about 1 month or more after ablation. The acute phase model can be described as a model having a collapsed blood circulation. The chronic phase model is in a state of decreased cardiac reserved force. Between the acute phase model and the chronic phase model, there is no significant difference in electrophysiological characteristics such as body surface electrocardiogram (ECG), monophasic action potential (MAP), monophasic action potential duration (MAP90), effective refractory period (ERP) and action potential terminal period (TRP).

The chronic phase model, compared to normal monkeys or the acute phase model, exhibits cardiac dilation and has a significantly higher concentration of ANP (atrial natriuretic peptide) or BNP (Brain natriuretic peptide) in the blood. Hence, the chronic phase model of the present invention can also be used as a chronic heart failure model. The concentration of ANP or BNP in the blood of the chronic phase model of the present invention is about 2 to 50 times, preferably about 5 to 20 times, higher than that in normal monkeys.

The model animal of the present invention has the concentration of noradrenaline in the blood significantly elevated compared to normal monkeys. From this, the model animal of the present invention can also be used as a model of sympathetic hypertonia. The concentration of noradrenaline in the blood of the model animal of the present invention is higher about 1.5 to 5 times, preferably about 2 to 3 times, than that in normal monkeys.

The proarrhythmia model animal of the present invention is a model in which even if arrhythmia develops with administration of a drug and the like, it does not become fatal and recovery occurs, unlike model animals prepared with other species such as the dog. Hence, the model animal of the present invention can be repeatedly utilized by waiting a recovery from arrhythmia, and then administering the next drug. Time to recovery varies depending on the kind and dose of the drug administered, and is normally 1 day to 2 weeks.

The present invention provides a method of generating a proarrhythmia model animal, comprising a step for inserting an electrode catheter to the heart of a monkey, and ablating the atrioventricular node with the catheter.

The monkey used is as described above, and is not subject to limitations as to age, sex and the like, but because of the capability of surviving for a long time as a model animal, a monkey, preferably a cynomolgus monkey, at 1 to 10 years of age is particularly used.

In the foregoing ablation step, any electrode catheter in common use in the art can be used without limitation; in the case of a cynomolgus monkey, one having a size of 5 to 6 French is preferable.

As the portion for insertion of the electrode catheter, the femoral vein, femoral artery, cubital vein or external jugular vein and the like can be mentioned; usually, it is preferable to insert the electrode catheter from the right femoral vein. For example, first, the monkey is anesthetized with pentobarbital or halothane or the like, and to stabilize the respiration, the monkey is intubated, and oxygen or the atmosphere is supplied in a given amount (for example, 10 to 20 ml/kg) from an artificial ventilator. After the respiration of the monkey is thus stabilized, an electrode catheter furnished with an electrode attached to the tip thereof is inserted from the femoral vein to the atrioventricular node region, and the tip electrode is immobilized at the specified position. Next, from the electrode catheter, a high-frequency electric current (for example, 500 kHz, 20 W) is applied to the atrioventricular node region for about 15 seconds or more (e.g., 30 seconds to 1 minute) to cauterize and destroy the atrioventricular node, whereby an atrioventricular block monkey is prepared. In addition to those exemplified above, ablation conditions can be set as appropriate, according to the species and age of the monkey used, and the like.

Using the proarrhythmia model animal thus obtained, the QT interval prolongation by a drug can be evaluated. The present invention provides such a method.

The method of evaluating the QT interval prolongation by a drug preferably comprises the following steps:

(1) a step for administering a drug to the foregoing model animal,
(2) a step for measuring the electrocardiogram QT interval or QTc interval in the recipient animal, and comparing the same with the electrocardiogram QT interval or QTc interval in the same recipient animal but before administration, and
(3) a step for evaluating the potential possibility of the QT interval prolongation by the drug on the basis of the results obtained in the foregoing comparison step.

### Step (1)

As the drug, any drug for evaluating QT interval prolongation can be used. If it is intended to demonstrate that the drug does not exhibit QT interval prolongation action or proarrhythmia action, this is not limiting. Regarding the dose of the drug, it is confirmed that a drug known to prolong the QT interval induces QT interval prolongation in the model animal of the present invention, and a range of the dose of the subject of evaluation can be set on the basis thereof. The maximum dose is preferably about 1 to 30 times the maximum clinical daily dose. Although the method of administration can be chosen as appropriate according to the drug, it is preferably the same as the method of administration to humans in clinical settings.

### Step (2)

A measurement of QT interval or QTc interval can be performed for an appropriate time according to the drug administered, after completion of the step (1), and is normally 1 to 24 hours. By attaching a Holter electrocardiograph to a model animal, monitoring for a long time is possible.

QT interval indicates the time interval from the start of the electrocardiogram Q wave to the end of the T wave, and is normally expressed in ms. QTc interval (ms) is a value obtained by correcting the fluctuation of QT interval due to heart rate by a numerical formula, and can be obtained by the following equation.

QTc=QT÷³√ (60÷ventricular rhythm)

Here, ventricular rhythm refers to the frequency of excitation of the ventricle in a complete atrioventricular block, and the unit of measurement is beat/minute. Shown above is the correction formula reported by Fridericia (reference: Fridericia, L. S., 1920. Die systolendauer in elektrokardiogramm bei normalen menschen und bei herzkranken. Acta Med. Scand. 53, 469-486), which, however, is not to be construed as limiting.

Next, a comparison is made between the QT interval or QTc interval in the recipient animal and the QT interval or QTc interval in the same animal but before administration. QT interval or QTc interval prolongation can be determined by (value after administration)−(value before administration).

### Step (3)

If the foregoing comparison reveals a significantly prolonged QT interval or QTc interval after administration of the drug, the drug administered can be judged as a drug involving a risk for onset of long QT interval syndrome. For a drug thus judged, transition to clinical studies can be prematurely discontinued.

Using the model animal of the present invention, a candidate substance possessing antiarrhythmic action can be screened for; the present invention provides such a screening method (screening method I).

### (Screening Method I)

The candidate substance may be any commonly known substance or novel substance; for example, a nucleic acid, glucide, lipid, protein, peptide, organic low molecular compound, a compound library prepared using combinatorial chemistry technology, a random peptide library prepared by solid phase synthesis or the phage display method, or naturally occurring ingredients derived from microorganisms, animals, plants, marine organisms and the like, and the like can be mentioned.

A judgment to determine whether or not the candidate substance possesses antiarrhythmic action is made as described below. The candidate substance is administered to the model animal of the present invention by a method appropriate for administration of the substance, a drug known to cause arrhythmia (positive control) is administered to the model animal before, simultaneously with, or after the candidate substance, and electrocardiogram is taken. By checking the presence or absence of onset of arrhythmia on the electrocardiogram, a candidate substance that suppresses the onset is selected. It is preferable that the time and severity of arrhythmia caused when the positive control alone is administered be measured by electrocardiogram and comprehended for control.

The positive control used in the screening method I is not subject to limitation; for example, some of group Ia or group III antiarrhythmic drugs, and some of antibiotics, antifungal drugs, anti-allergic drugs, antihyperlipemic drugs, antipsychotic drugs, tricyclic antidepressants, anticancer agents, gastrointestinal function promoters and the like can be mentioned; specifically, dl-sotalol, cisapride, astemizole, haloperidol, moxifloxacin, terfenadine (combination of terfenadine and ketoconazole) and the like can be mentioned.

The model animal of the present invention is also useful as a chronic heart failure model in the chronic phase; using this model, a candidate substance that ameliorates chronic heart failure can be screened for; the present invention provides such a screening method (screening method II).

### (Screening Method II)

The candidate substance used may be the same substance as in the screening method I.

A judgment to determine whether or not the candidate substance ameliorates chronic heart failure is made as described below. The candidate substance is administered to the model animal of the present invention by a method appropriate for administration of the substance. Because a substance that can become a therapeutic drug for chronic disease is targeted, administration of the candidate substance is desirably performed for a long time. The concentration of ANP and/or BNP in the blood, which is an index of chronic heart failure, is measured, and a candidate substance that significantly reduces the concentration of ANP and/or BNP compared to before administration after elapse of a given time is selected.

The model animal of the present invention has accentuated tension of the sympathetic nerve; using this model, a candidate substance that ameliorates sympathetic hypertonia can be screened for; the present invention provides such a screening method (screening method III).

### (Screening Method III)

The candidate substance used may be the same substance as in the screening method I.

A judgment to determine whether or not the candidate substance ameliorates sympathetic hypertonia is made as described below. The candidate substance is administered to the model animal of the present invention by a method appropriate for administration of the substance. The concentration of noradrenaline in the blood, which is an index of sympathetic hypertonia, is measured, and a candidate substance that significantly reduces the concentration thereof compared to before administration is selected.

### EXAMPLES

The present invention is hereinafter described in detail by means of the following Examples, which, however, are not to be construed as limiting the scope of the invention.

The following experiments were properly performed at Ina Research Inc. (2148-188, Nishiminowa, Ina-shi, Nagano) in compliance with the “The Law for Partially Amending The Law for the Humane Treatment and Management of Animals” (Jun. 22, 2005, Law No. 68) and the “Ina Research Inc. Animal Experiment Guideline” (amended on Jan. 1, 2004) per a study protocol reviewed by the company's Institutional Animal Care and Use Committee (IACUC). Ina Research Inc. has been certified by AAALAC International (certification number: 00107).

### Example 1

### Generation of Proarrhythmia Monkey Model

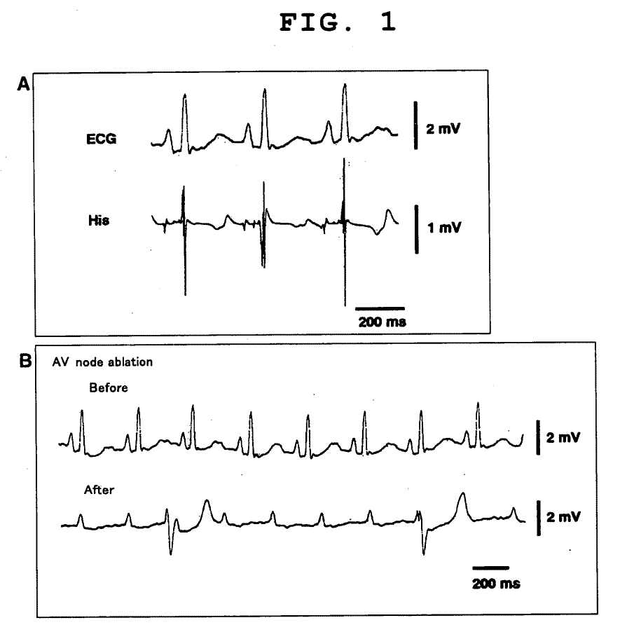

Pentobarbital was gradually administered by intravenous injection to a male or female cynomolgus monkey at about 4 years after birth (about 3 kg) (30 mg/kg), simultaneously the trachea was intubated, and oxygen or the atmosphere was supplied in a given amount (10 to 20 ml/kg) using an artificial ventilator to achieve respiratory management. After the thigh was shaven and disinfected with alcohol-soaked cotton, a guide wire was inserted to the femoral vein, and an electrode catheter (6 French size) furnished with a pacing electrode attached to the tip thereof was inserted from the femoral vein to the right ventricle. In search of a position that allows the highest level of recording of His bundle electrocardiogram, the tip electrode was immobilized, and intracardiac electrocardiogram (His) was measured (FIG. 1A). At the same time, body surface electrocardiogram (ECG) was also measured (FIG. 1A). Next, from the tip electrode of the electrode catheter, a high-frequency electric current (500 kHz, 20 W) was applied to the atrioventricular node region for 60 seconds to electrically cauterize the atrioventricular node, whereby the atrioventricle was blocked. Body surface electrocardiogram after ablation was measured, and the results of a comparison with the electrocardiogram before ablation are shown in FIG. 1B.

### Example 2

### Evaluation of Cardiac Dilation in Proarrhythmia Monkey Model

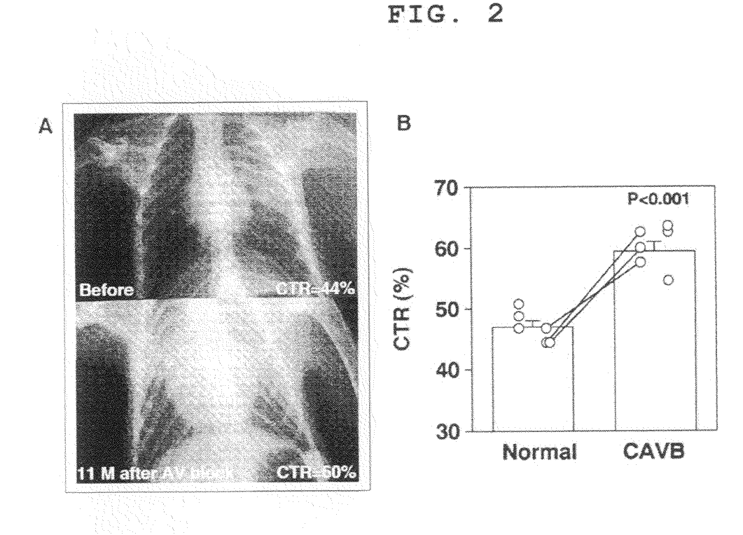

The chests of male or female cynomolgus monkeys were radiographed, and the cardiothoracic ratios were measured. Next, ablation was performed, the chests of cynomolgus monkeys after elapse of about 12 months were radiographed, and the cardiothoracic ratios were compared. The calculation formula used was maximum transverse diameter of heart÷maximum transverse diameter of thoracic cavity×100. The results are shown in FIG. 2.

FIG. 2A shows a case in which the heart dilated due to surgery for making a complete atrioventricular block; the cardiothoracic ratio changed from 44% to 60%. FIG. 2B shows mean values of cardiothoracic ratios in six cases, including three cases in which the evaluation was made on the same animal; the cardiothoracic ratio increased statistically significantly due to the complete atrioventricular block. From this, it was demonstrated that the heart dilated as a result of a compensation mechanism for heart failure due to the complete atrioventricular block.

### Example 3

### Physiological and Biochemical Examination of Proarrhythmia Monkey Model

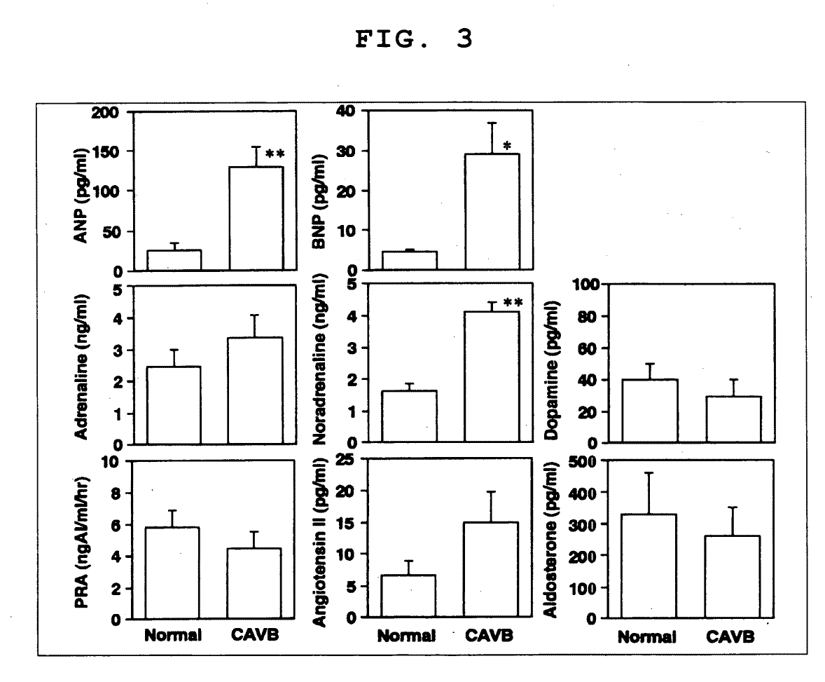

Blood was drawn from normal cynomolgus monkeys (Normal) and chronic atrioventricular block monkeys obtained in Example 1 (CAVB, monkey spending about 2 months after ablation), and the concentrations of Aldosterone, Angiotensin II, PRA, Adrenaline, Noradrenaline, Dopamine, ANP, and BNP in the blood were measured according to conventional methods. The results are shown in FIG. 3.

As shown in FIG. 3, the chronic atrioventricular block monkeys had significantly higher values of noradrenaline, ANP and BNP than those in the normal monkeys. From this, it was found that the monkey model of the present invention had the sympathetic nervous system in an accentuated state, and had a sign of chronic heart failure.

### Example 4

### Electrophysiological Evaluation of Proarrhythmia Monkey Model

An electrophysiological evaluation in the acute phase (immediately after ablation) and the chronic phase (2 months after ablation) was performed on the monkey model obtained in Example 1. Limb second lead electrocardiogram was recorded under pentobarbital anesthesia. From the femoral vein, a catheter electrode for monophasic action potential recording and pacing (1675P, manufactured by EP Technologies) was indwelled in the right ventricle, and monophasic action potential was recorded. Electrocardiogram was amplified using an electrocardiogram amplifier (AC-611G, manufactured by Nihon Kohden Corporation), and monophasic action potential was amplified using a DC pre-amplifier (300, manufactured by EP Technologies), and signals were recorded on a monitor (VC-604G, manufactured by Nihon Kohden Corporation). Cardiac pacing was achieved using a cardiac stimulator (SEC-3102, manufactured by Nihon Kohden Corporation), with the ventricle stimulated at 1 to 2 V, levels about doubling the stimulation threshold value.

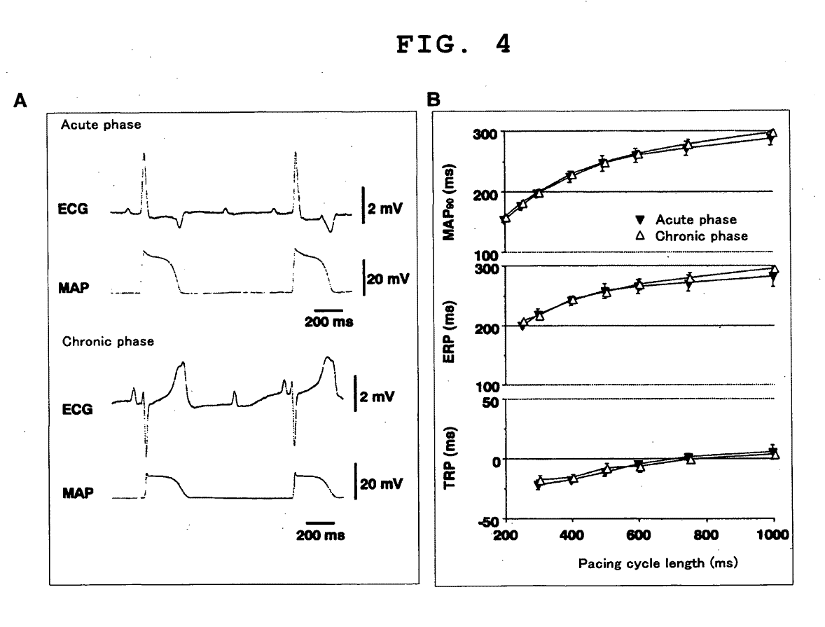

The results are shown in FIG. 4. FIG. 4A shows typical examples of body surface electrocardiogram (ECG) and monophasic action potential (MAP) in the acute phase just after conduct of atrioventricular node ablation and the chronic phase. FIG. 4B is a graph summarizing monophasic action potential duration (MAP90), effective refractory period (ERP) and action potential terminal period (TRP) for each pacing cycle length in the acute phase and chronic phase. These results show that no differences are observed in the electrophysiological properties of the ventricular muscle of the monkey model of the present invention between the acute phase and the chronic phase.

### Example 5

### Evaluation of QT Interval Prolongation by Dl-sotalol

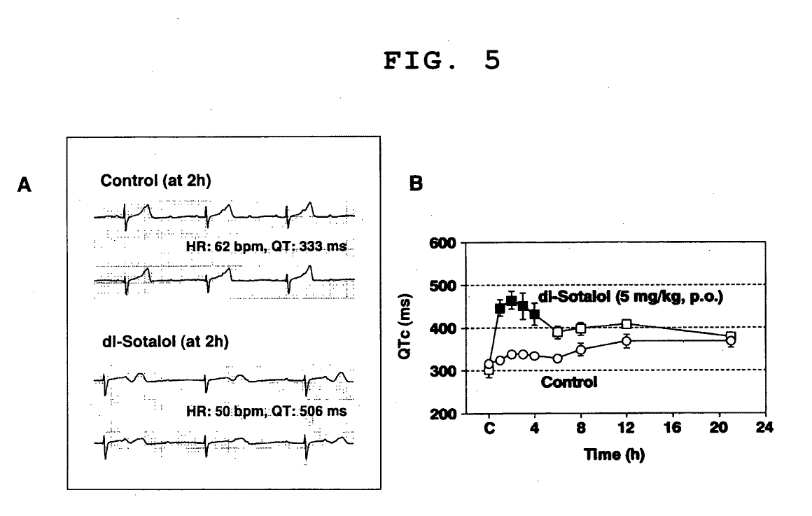

Using chronic atrioventricular block monkeys obtained in Example 1, electrocardiogram was recorded using a Holter electrocardiograph for 24 hours. As the control solution, 0.5% methylcellulose solution was orally administered, and changes of electrocardiogram were examined; on a later day, 5 mg/kg dl-sotalol (group-3 antiarrhythmic agent) was orally administered to the same animals, and electrocardiogram was measured. The results are shown in FIG. 5.

As shown in FIG. 5, QTc interval prolongation was observed with oral administration of 5 mg/kg dl-sotalol; 1 to 4 hours later, statistically significant action was observed compared to the solvent group. From this, it was found that the 5 mg/kg dl-sotalol was a dose that sufficiently prolonged the QT interval.

### Example 6

### Examination for Onset of Torsades de Pointes (TdP) with Dl-sotalol Administration

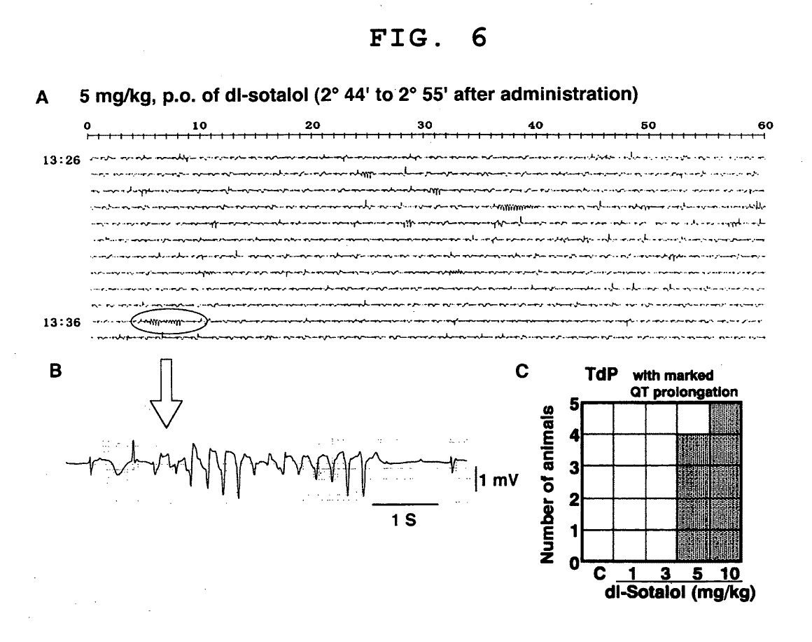

A Holter electrocardiograph was attached to each of five animals of the chronic atrioventricular block monkey model obtained in Example 1; 1, 3, 5 or 10 mg/kg dl-sotalol was orally administered to each animal, and electrocardiogram after administration was monitored. The results are shown in FIG. 6.

FIG. 6A shows an example electrocardiogram obtained with oral administration of 5 mg/kg dl-sotalol. The enlarged electrocardiogram shown in FIG. 6B represents a typical case of TdP; several similar arrhythmias developed during the 11-minute period indicated. A feature of the TdP occurring in the chronic atrioventricular block monkey model is that all episodes cease spontaneously. FIG. 6C summarizes the number of episodes of TdP that occurred in the five animals receiving the various doses of sotalol. Although TdP occurred in 4 of the 5 animals at 5 mg/kg and all animals at 10 mg/kg, all episodes ceased spontaneously; no animals experienced progression to ventricular fibrillation and death. From this, it was found that the chronic atrioventricular block monkey model, unlike the model using the dog, could be repeatedly utilized for drug evaluation.

### INDUSTRIAL APPLICABILITY

Because the model animal of the present invention is an animal obtained by ablation of the atrioventricular node of a monkey, it can serve as a model having a heart shape and pharmacokinetics closest to those of humans. The model animal of the present invention is a model exhibiting an atrioventricular block so that arrhythmia is easy to induce. By providing such a model animal, it becomes possible to accurately evaluate the onset of long QT syndrome induced by a candidate drug in the nonclinical study phase. Other model animals prepared from non-monkey species experience fatal arrhythmia, whereas the model animal of the present invention unexpectedly allows a recovery from arrhythmia. Therefore, the valuable model animal can be effectively utilized, the same model animal can be repeatedly used for evaluation studies, and evaluation results with no variation due to individual differences can be obtained. By utilizing this feature, results of a study of multiple drugs or multiple studies of a single drug can be compared using the same criteria (the same animal). When a cynomolgus monkey is used as the source of the model animal, because its physical constitution (body weight) and heart size are smaller than those of model animals such as dogs, it is possible to reduce the amount of drug used for the evaluation, which leads to cost saving. Furthermore, the model animal of the present invention can also be utilized as a chronic heart failure model or a model of sympathetic hypertonia.

According to the method of the present invention for generating the model animal, it becomes possible to securely provide the foregoing model animal. According to the evaluation method of the present invention, it becomes possible to accurately evaluate the potential possibility of long QT syndrome induced by a candidate drug at the nonclinical study stage. According to the screening method of the present invention, a candidate substance possessing antiarrhythmic action, a candidate substance that ameliorates chronic heart failure, or a candidate substance that ameliorates sympathetic hypertonia can be significantly selected.

This application is based on a patent application No. 2005-315434 filed in Japan on Oct. 28, 2005, the contents of which are incorporated in full herein by reference.

1. A proarrhythmia model animal of a monkey, which is generated by ablating the atrioventricular node.

2. The model animal of claim 1, wherein the atrioventricular node is blocked.

3. The model animal of claim 1, wherein the ablation is conducted by electrical stimulation from the tip of a catheter.

4. The model animal of claim 1, which is an acute phase model less than 1 month after ablation.

5. The model animal of claim 1, which is a chronic phase model 1 month or more after ablation.

6. The model animal of claim 5, which is a chronic heart failure model.

7. The model animal of claim 6, wherein the concentration of atrial natriuretic peptide or cerebral natriuretic peptide in the blood is elevated compared to a normal monkey.

8. The model animal of claim 1, which is a model of sympathetic hypertonia.

9. The model animal of claim 8, wherein the concentration of noradrenaline in the blood is elevated compared to a normal monkey.

10. The model animal of claim 1, wherein the monkey is a cynomolgus monkey.

11. A method of generating a proarrhythmia model animal, comprising a step for inserting an electrode catheter to the heart of a monkey, and ablating the atrioventricular node with the catheter.

12. The generating method of claim 11, wherein the size of the catheter is 5 to 6 French.

13. The generating method of claim 11, wherein the monkey is a cynomolgus monkey.

14. A method of evaluating the QT interval prolongation by a drug, comprising using the model animal of claim 1.

15. A method of evaluating the QT interval prolongation by a drug, comprising:
a step for administering the drug to the model animal of claim 1,
a step for measuring the QT interval or QTc interval in the recipient animal, and comparing the same with the QT interval or QTc interval in the same animal before administration, and
a step for evaluating the potential possibility of the QT interval or QTc interval prolongation by the drug on the basis of the results obtained in the comparison step.

16. A screening method for a candidate substance possessing antiarrhythmic action, comprising using the model animal of claim 1.

17. A screening method for a candidate substance that ameliorates chronic heart failure, comprising using the model animal of claim 6.

18. A screening method for a candidate substance that ameliorates sympathetic hypertonia, comprising using the model animal of claim 8.

19. A proarrhythmia model animal of a monkey, wherein the monkey possesses an atrioventricular block, and the concentration of atrial natriuretic peptide or cerebral natriuretic peptide in the blood is elevated compared to a normal monkey.

20. The model animal of claim 19, wherein the concentration of atrial natriuretic peptide or cerebral natriuretic peptide in the blood is elevated about 2 to 50 times compared to a normal monkey.

21. The model animal of claim 19, wherein the concentration of noradrenaline in the blood is elevated compared to a normal monkey.

22. The model animal of claim 21, wherein the concentration of noradrenaline in the blood is elevated about 1.5 to 5 times compared to a normal monkey.

23. The model animal of claim 21, which is a model of sympathetic hypertonia.

24. The model animal of claim 19, which is a model concurrently suffering cardiac hypertrophy and cardiac dilation that accompany volume overload.

25. The model animal of claim 19, wherein the monkey is a cynomolgus monkey.

26. The generating method of claim 12, wherein the monkey is a cynomolgus monkey.
